(19) United States
(12) Patent Application Publication
(10) Pub. No.: US 2008/0091090 A1
(43) Pub. Date: Apr. 17, 2008

(54) Self-contained surface physiological monitor with adhesive attachment

(76) Inventors: Kenneth Shane Guillory, Salt Lake City, UT (US); Dimitri Yatsenko, Salt Lake City, UT (US)

Correspondence Address: THORPE NORTH & WESTERN, LLP., P.O. Box 1219, SANDY, UT, 84091-1219, US

(21) Appl. No.: 11/827,387

(22) Filed: Jul. 9, 2007

Related U.S. Application Data
(60) Provisional application No. 60/829,148, filed on Oct. 12, 2006.

(51) Int. Cl. A61B 5/00

(52) U.S. Cl. 600/301

(57) ABSTRACT

A self-contained monitor device to monitor at least one physiological parameter of a subject includes a pad with an adhesive layer configured to adhere to a subject's skin. The pad carries at least one physiological sensor, a signal processing means, an indicator, and a power source.

### PRIORITY CLAIM

Priority of U.S. Provisional Patent Application Ser. No. 60/829,148 filed on Oct. 12, 2006, is claimed; and which is herein incorporated by reference.

This is related to U.S. patent application Ser. No. ______, filed Jul. 9, 2007, as TNW Docket No. 2517-002 entitled “Single Use, Self-Contained Surface Physiological Monitor”; U.S. patent application Ser. No. ______, filed Jul. 9, 2007, as TNW Docket No. 2517-004 entitled “Self-Contained Seizure Monitor and Method”; which are herein incorporated by reference.

### BACKGROUND

1. Field of the Invention

The present invention relates generally to a self-contained device to monitor at least one physiological parameter of a subject.

2. Related Art

It can be difficult to monitor or diagnose medical or physiological conditions of a patient away from a medical facility. Often, medical equipment is tied to use in such a facility requiring transport of the patient to the facility. In some situations, special vehicles can transport some special equipment to a patient. It will be appreciated, however, that situations can be presented in which transportation of the patient may not be an option, or in which immediate medical attention is required without waiting for transportation, or when conventional monitoring equipment cannot be supplied in sufficient quantities for the numbers of patients requiring monitoring.

For example, it can be difficult to assess if unconscious or semi-conscious patients are having nonconvulsive seizures, especially in situations where nerve agents may have been used and patients are experiencing extreme muscle fatigue and/or partial paralysis. The ability to robustly and efficiently identify status epilepticus (SE) in these patients can greatly assist emergency medical personnel in determining initial treatment on site and during transport to a medical facility where more comprehensive EEG monitoring will be performed.

### SUMMARY OF THE INVENTION

It has been recognized that it would be advantageous to develop a device to monitor at least one physiological parameter of a subject that is self-contained. In addition, it has been recognized that it would be advantageous to develop a monitor device to monitor at least one physiological parameter of a subject that is single-use, or disposable. In addition, it has been recognized that it would be advantageous to develop a monitor device to monitor at least one physiological parameter of a subject with a graphical display capable of displaying a physiological variable value as an instantaneous value or as a trace showing the evolution of the condition in time.

The invention provides a self-contained monitor device configured to monitor at least one physiological parameter of a subject. A pad has an adhesive layer configured to adhere to a subject's skin. At least one physiological sensor is carried by the pad and is configured to sense at least one subject physiological parameter and generate a physiological signal. A signal processing means is carried by the pad and is operatively coupled to the at least one physiological sensor for processing the physiological signal. At least one indicator is carried by the pad and is electrically coupled to the signal processing means and is configured to indicate information associated with the pad, the physiological parameter, the subject, or combinations thereof. A power source is carried by the pad and is electrically coupled to the at least one physiological sensor, the signal processing means and the indicator.

In addition, the invention provides a method for monitoring a physiological parameter of a subject, comprising:

adhering an adhesive layer carried by a pad of a self-contained monitor device on skin of a subject;

causing the monitor device to power from a power source carried by the pad of the self-contained monitor device, and causing at least one physiological sensor carried by the pad and to sense at least one subject physiological parameter and generate a physiological signal, and causing a signal processor carried by the pad and coupled to the at least one physiological sensor to process the physiological signal; and

perceiving an output of an indicator carried by the pad and electrically coupled to the signal processor.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a top perspective view of a self-contained monitor device introducing several types of indicators used in several embodiments of the present invention;

FIG. 2 is a schematic view of a self-contained monitor device in accordance with an embodiment of the present invention configured as a self-contained seizure monitor device displaying the evolution of epileptiform electrographic activity and also including pulse oximetry and heart rate monitoring;

FIG. 3 is a schematic view of the monitor device of FIG. 2 shown applied to a subject;

FIG. 4 is a top perspective view of an adhesive physiological monitor device according to another embodiment;

FIG. 5 is a schematic view of a patient or a subject showing possible locations for sensors of the device in FIG. 4;

FIG. 6 is a schematic view of the monitor device in FIG. 4 applied to a human subject;

FIG. 7 is a schematic circuit outline of the monitor device of FIG. 4;

FIG. 8 is a bottom perspective view of the monitor device in FIG. 4 shown with the release liner partially removed;

FIG. 9 is an exploded perspective view of the monitor device of FIG. 4;

FIG. 10 is a top perspective view of another self-contained monitor device in accordance with another embodiment including a means to limit the device to a single use;

FIG. 11 is a bottom perspective view of the monitor device in FIG. 10 with the release liner partially removed.

FIG. 12 is a schematic view of a monitor device including a separate physiological sensor applied adhesively;

FIG. 13 is a schematic view of a monitor device including a separate physiological clip-on sensor;

FIG. 14 is a schematic view of another self-contained monitor device in accordance with another embodiment of the present invention including both integrated and separate physiological sensors;

FIG. 15 is a schematic view of another self-contained monitor device in accordance with another embodiment of the present invention comprising a reusable portion and a disposable portion;

FIG. 16 is a schematic view of another self-contained monitor device in accordance with another embodiment of the present invention comprising multiple adhesive layers to enable multiple use;

FIG. 17 is a schematic view of a treatment kit including the self-contained monitor device;

FIG. 18 is a schematic view of a self-contained monitor device in wireless communication with an external device such as a hand-held computer;

FIG. 19 is a diagram showing the wireless system diagnostics and upgrade;

FIG. 20 is a schematic view of a patient simulator in accordance with an embodiment of the present invention.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

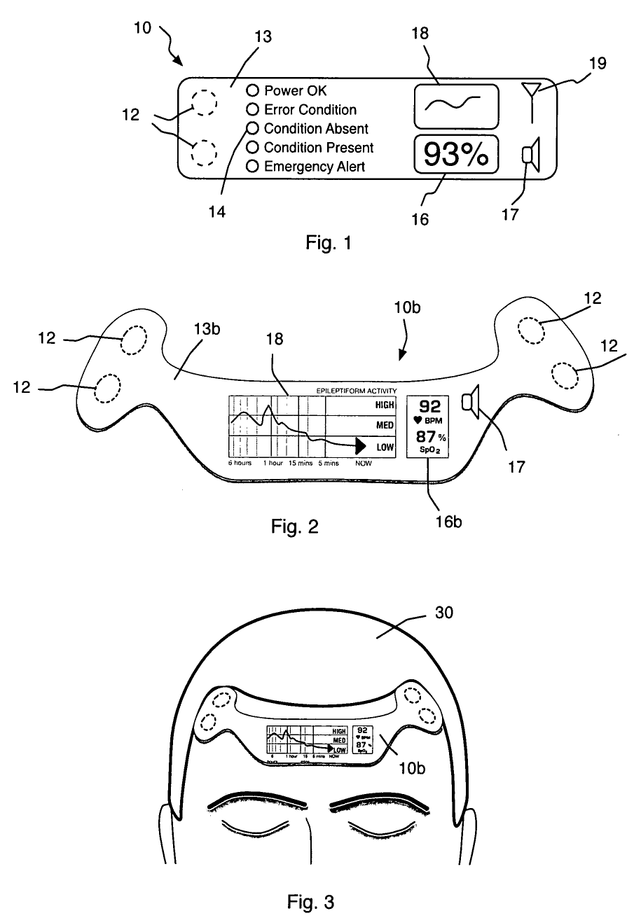

As illustrated in FIGS. 1-12, various embodiments of a self-contained monitor device, indicated generally at 10-10e, in accordance with an exemplary implementation of the present invention is shown to monitor at least one physiological parameter of a subject 30 (FIG. 3), such as a human patient. The device can monitor, and the physiological parameter can include, heart rate, oxygen level, respiration rate, body temperature, cholesterol level, blood glucose level, galvanic skin response, electrophysiology, blood pressure, EEG, ECoG, EMG, ECG, ENG, skin impedance, humidity, ultrasound absorption, light and infrared absorption, acoustic or vibratory signals, movement, combinations thereof, etc. Based on the physiological parameters measured, the device can determine health status, determine degree of injury, and/or detect the presence or lack of pathological conditions. In an embodiment, the device also indicates the progression of a physiological condition over time as a time series on a graphical display 18. The self-contained device can be completely integrated, topically applied, and disposed entirely on the subject.

In accordance with one aspect of the present invention, the monitor device 10 can include a pad, patch, or housing 13 that carries and/or contains various components of the device. The pad can be flexible and capable of contouring to a subject's body. Alternatively, the pad or housing can include rigid portions joined by flexible portions that allow the rigid portions to pivot with respect to one another to more closely contour to the subject's body. The pad can be formed of a plurality of layers stacked together to form the pad, as described in greater detail below. The pad can have a substantially flat configuration in storage, and an arcuate or deflected configuration in use. The various components can be integrated into the pad so that the pad or device can be topically applied and entirely disposed on the subject. The pad can be sized and shaped to cover and/or extend between desired portions of the subject's body. For example, the pad can have a length of approximately 4-6 inches if applied to a subject's forehead.

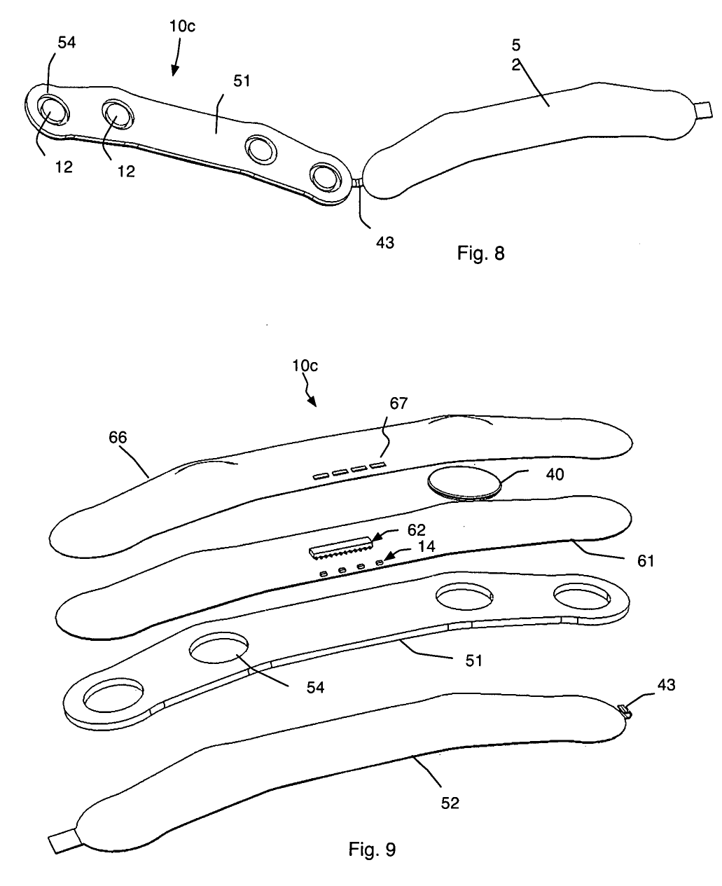

An adhesive or adhesive layer 51 (FIGS. 8 and 9) can be disposed on the device or pad to adhere the device or pad to a subject's skin. For example, the pad and adhesive layer can include single-sided or double-sided pressure sensitive adhesive foam. The adhesive layer or foam can form one of the plurality of layers of the pad. A release liner 52 (FIGS. 8 and 9) can be removably disposed over the adhesive layer 51 before use or during storage to protect and preserve the adhesive layer, and to resist unintended adhesion. Alternatively, the pad can be applied to the subject's skin by force, wrappings, suction, gravity, water tension, etc. The adhesive layer 51 can be an integrated part of the pad that can limit the device to a single use. For example, the adhesive layer can be configured with sufficient adhesion for a single use, with exposure to air and/or skin oil effectively prohibiting subsequent use. Alternatively, the device can be configured for multiple uses with the same or a different subject. For example, various components of the device can be removable from the pad or adhesive layer so that the same components can be used with another pad or another adhesive layer.

One or more physiological sensors 12 can be carried by the device or pad and configured to be applied to the subject's skin. Thus, the adhesive layer 51 can surround the sensors 12 to maintain contact between the skin and the sensors. In one aspect, one or more apertures 54 (FIGS. 8 and 9) can be formed in the adhesive layer 51 with the sensors 12 partially or wholly disposed within the apertures. It will be appreciated that an electrically conductive gel can be disposed over the sensors and protected by the release liner 51 and/or an adhesive seal. In addition, a thin film of sodium chloride can coat the sensors to draw moisture into the electrode interfaces and thus improve contact through oily skin.

The sensors 12 can be any type of sensor or electrode and can be active or selectively active depending on the state of the device and the type of analysis being performed. The sensors can passively sense physiological signals, as in the case with EEG electrodes, or can actively apply energy to the subject to sense the signal or parameter, such as with electrical impedance measurement or light absorption measurement for blood oxygenation. Active sensing can also include applying visual, auditory, somatosensory or electrical stimulation to record electrophysiological measures such as nerve conduction velocity or evoked responses such as ABER or P300 waveforms. The electrodes may be made of Ag/Ag Cl packaged with an electrically conductive gel and a special adhesive sealed cover to prevent the gel from drying out. The electrodes may also be dry gold electrodes coated with a thin film of sodium chloride to quickly draw moisture into the electrode interfaces and improve contact through oily skin. The electrodes may also be made of another electrically conductive material.

The sensors can sense or monitor one or more subject physiological parameters and generate physiological signals. As described above, the sensors can sense or monitor heart rate, oxygen level, respiration rate, body temperature, cholesterol level, blood glucose level, galvanic skin response, electrophysiology, blood pressure, EEG, ECoG, EMG, ECG, ENG, skin impedance, humidity, ultrasound absorption, light and infrared absorption, acoustic or vibratory signals, movement, combinations thereof, etc. The sensors can be configured to sense the same or different physiological parameters, or different aspects of the same physiological parameter.

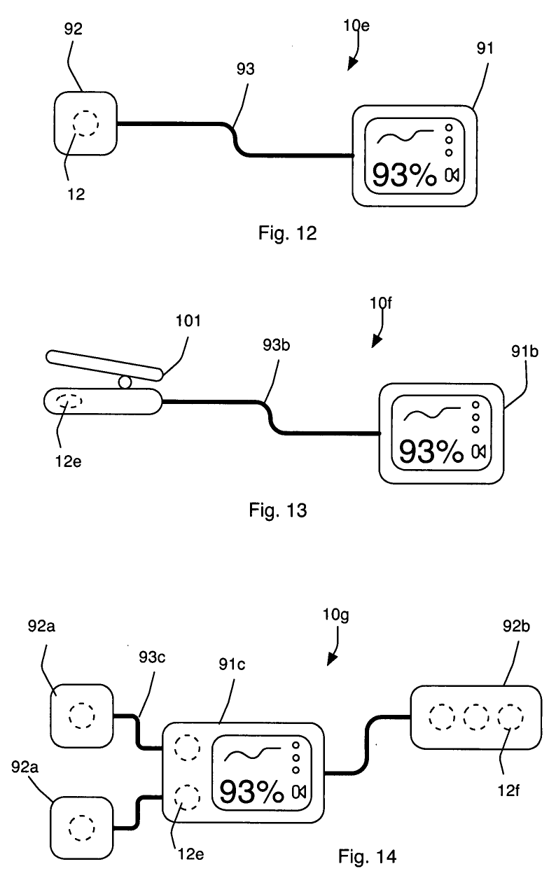

As described above, the sensors can be integrated into the pad or housing as one unit applied to the patient. Alternatively, one or more sensors can extend from the main unit and be coupled to the main unit by tabs or lead wires. Thus, the sensors can be disposed on other parts of the subject away from the main unit (FIGS. 12-14).

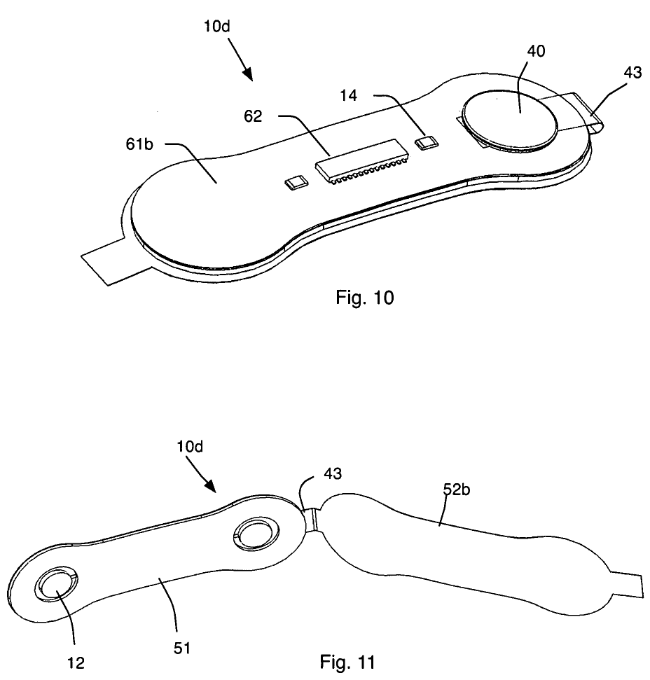

Signal processing unit or units 62 (FIGS. 9 and 10) or other electronics, integrated circuits or signal processors can be carried by or contained within the device or pad. The signal processing units 62 can be coupled to the one or more physiological sensors 12. The signal processing units 62 can process or analyze the physiological signals received from the sensors and generate other signals, such as display or indicator signals or alarms. The signal processing units 62 and electrical connections can be disposed on a circuit layer 61 such as a thin-film polyimide (Kapton) circuit substrate that is flexible. This circuit layer may contain all the necessary electronics in the patch. The circuit layer may 61 can be disposed on top of the adhesive layer 51 or the double-sided pressure sensitive adhesive foam.

The signal processing units 62 can analyze signals from the sensors. Analysis can be performed by digitally processing the signals in a computing device such as a microprocessor, DSP, FPGA, or CPLD device, including any multiplexing and/or analog to digital conversion that may be necessary for processing the signals in the digital domain. Analysis may also be performed by applying analog implementations of algorithms, computational techniques, or detection methods, including linear and non-linear filtering, rectification, summation, logarithm/exponential conversion, thresholding, comparison, etc.

The integrated circuit and signal processor can also include internal programs and settings. The programs and settings can be reprogrammed, changed and/or updated by exchanging data with the device through an electrical contact, inductive link, optical and/or infrared link, RF data link, Bluetooth or other wired or wireless method that can be applied for electronic communication. The device can include error checking and/or correction schemes for validating the data exchanged such as CRC, checksum, and other known techniques, and/or include a variety of known authentication methods for verifying the identity of the programmer and authorization to change the device. Data exchanges with the system can be performed with direct access to the system, through external device packaging, through special windows or access ports within packages, or through packages that include kits or other components used with the system.

The signal processing units 62 can process or analyze signals from the sensors 12, and can generate a physiological result or value. The signal processing units 62 can generate a display signal for a visual or audio indicator or a graphical display. The physiological parameter or value can be heart rate, oxygen level, respiration rate, body temperature, cholesterol level, blood glucose level, galvanic skin response, electrophysiology, blood pressure, EEG, ECoG, EMG, ECG, ENG, skin impedance, humidity, ultrasound absorption, light and infrared absorption, acoustic or vibratory signals, movement, combinations thereof, etc.

In addition, the integrated circuit can generate a physiological condition index based on at least one physiological parameter. For example, the integrated circuit can generate an epileptiform activity index or a status epilepticus index, such as high, medium or low. The indicator or graphical display can display the physiological condition index.

Furthermore, the signal processing units can generate an alarm signal in response to a change of the physiological condition index. The alarm signal can be send to an indicator, such as a LED or graphical display, or to an audible device, such as a speaker or buzzer.

In addition, the signal processing units can generate other signals based on the operation of the device, such as power on, battery level, sensors operable, etc. Furthermore, the integrated circuit can generate user prompts or instruction signals for the indicator, such as prompting the user to administer medication, etc. The integrated circuit or signal processor is one example of a signal processing means for processing the physiological signal or for processing a signal from the at least one physiological sensor.

One or more indicators, such as LED indicators 14, numeric displays 16 or 16b, audible indicators 17 or speakers, or graphical displays 18 can be carried by the device 10 and electrically coupled to the signal processing units 62, such as by conductive traces or lines on the circuit substrate. The indicator can include one or more lights or LEDs 14, or can be numeric displays 16 such as custom LCD, or can be graphical displays 18, such as LCD or organic LED screens. Indicia can be disposed on the pad adjacent the one or more lights or LEDs to indicate the condition of the light or LED. The indicator 14, or the LEDs or LCD, can be carried by the circuit substrate 61, and visible through a cover layer 66 (FIG. 9), or aperture 67 (FIG. 9) therein, as described in greater detail below. The indicators 14, 16, 17, and 18 can indicate or display information associated with the pad, the physiological parameter, the subject, or combinations thereof. In addition, the indicators 14 can also double as a switch or button, such as a push button LED. Furthermore, the indicator can be a graphical display capable of displaying graphical information, such as the physiological value or its progression in time.

The indicator can also be, or can include, simple value indicators, such as alphanumeric displays, bar meters, light indicators with intensity or color modulation, and/or other quantitative displays commonly used for electronic instruments, such as LEDs, LCDs, electroluminescent, organic LEDs, mechanical displays, cholesteric LCDs, electronic paper, etc. In addition, the indicator can also be, or can include, auditory indicators, beeps, alarms, quantitative indicators, such as auditory tones, beep rates, etc, that change in tone and/or frequency, or even speech signals that report information or give verbal prompts to users. The indicator can also include indicators of the presence or lack of specific subject or patient conditions or dangerous parameter ranges by state indicators and/or binary true/false type indicators that are either present or not. The indicator can also include indicators of system status including battery level, power, sensor conditions, analysis progress, or other information to update the user on condition or state of the system. The indicator can also include error signals used to instruct the user to correct the application and/or use of the device or pad. The indicator can also provide reliability or confidence level information for analyzed data to assist the user in interpreting the results.

In situations where the system is used in kits that include other components, such as devices or drugs, the displays may also reference specific kit components or kit component labels, and/or indicate the need to apply specific kit components based on analysis performed. The kit can also contain detailed instructions on how to administer the drugs.

A power source 40 (FIGS. 9 and 10), such as a battery, can be carried by the device 10 or pad and electrically coupled to the physiological sensors 12, the signal processing units 62 and the indicators 14, 16, 17, or 18. The power source 40 or battery can be carried by the circuit layer 61. In addition, the power source 40 can be sealed within the device 10 or pad so that the power source is non-replaceable or non-removable.

The power source 40 can be, or can include, an integrated or replaceable energy source such as a battery, fuel cell, capacitor, dynamo, or other electromechanical system that derives electrical power from stored mechanical energy such as a spring or pressure tank. The device or power source can also receive power externally from galvanic coupling to the skin, light and/or solar power, chemical fuel, external inductive power, or mechanical movement that is converted to electrical power for powering the device. The device or power source can also contain an energy storage device that uses the described external sources to charge and/or recharge the device, for example, adding fuel to a fuel cell, charging an integrated capacitor by inductive power, etc.

A cover 66 (FIG. 9) or cover layer can be disposed over the circuit layer 61, the signal processing units 62, the indicators 14, 16, 18, the power source 40, or combinations thereof. The cover can be formed of a polymeric material, such as an acrylic, and can have an adhesive bottom to secure to the pad. In addition, the cover 66 can include apertures 67 (FIG. 9) through which the indicator 14, 16, 16b and/or 18 can extend or can be viewed, or through which buttons or other input can extend or be accessed. The cover can be substantially flat with raise portions to accommodate the power source, integrated circuit, sensors, or combinations thereof. The apertures 67 can be covered with a clear film to allow viewing of the indicators while maintaining integrity to moisture.

The device 10 or pad can be formed by the various layers, such as the adhesive layer 51, the circuit layer 61 and the cover layer 66. The layers can include adhesive or can be adhered together. It will be appreciated that other forms of joining the layers can be used, such as sonic welding, etc.

An exploded diagram of the general assembly concept for the device is shown in FIG. 9. The core of the assembly is a very flexible thin-film Kapton circuit assembly with top and bottom copper layers. The electrodes are on the bottom of the substrate and the electronics will be surface mounted on the top. The top/bottom circuit layers also include actively driven shields over the electrode areas to reduce electrical interference and motion artifacts. The system can use dry gold electrodes for patient contact. These can be coated with a thin film of sodium chloride to quickly draw moisture into the electrode interfaces and improve contact with the skin. Wet electrodes (using paste or gel) currently dominate in clinical EEG applications as they have a longer history of use and they can make better contact through hair. However, controlled studies show that, when used with proper electrical shielding, dry metal electrodes provide a more robust connection that is more immune to electrical and movement artifact (A. Searle and L. Kirkup, “A direct comparison of wet, dry and insulating bioelectric recording electrodes”, Physiol. Meas. 21(2000) 271-283.). In applications where motion artifacts are a significant problem, a 3-axis accelerometer can be included in the device for adaptive motion artifact cancellation.

The layers, or substrates forming the layers, can be substantially flexible. For example, the pressure sensitive adhesive foam of the adhesive layer, the polyimide (Kapton) circuit substrate of the circuit layer, and the acrylic material of the cover layer can be substantially flexible, and the combined adhered layers can be substantially flexible. It will be appreciated that the power source, integrated circuit and sensors can be rigid, and can create rigid portions of the pad, while the spaces between the rigid portions can be flexible portions about which the rigid portions pivot. In addition, the pad 13 or housing can be sealed, or one or more of the components can be sealed within the pad or the housing. For example, the power source 40 or battery can be sealed within the pad 13, or between the cover layer 66 and the circuit layer 61 or between other layers to resist or prevent removal of the power source. Resisting access to the battery can limit the device to a single use, as described in greater detail below.

The device can also include a button, switch or other activator capable of activating the power source or the device for use. For example, power can be enabled by a switch that is closed or an energy barrier that is broken by the user activating a control, removing a part, removing the device from packaging, removing adhesive backings or strips, and/or applying the device to the skin. For example, a tab 43 (FIGS. 9-11) can extend between the power source 40 or battery, and an electrical connection, such as on the circuit layer. The tab can physically block or prevent the power source from electrically connecting to the circuit layer, or the rest of the device. Removing the tab can allow the electrical connection, and thus operation of the device. In addition, the tab 43 can be coupled to the release liner 52 and 52b (FIGS. 9-11) such that removal of the release liner of the device also removes the tab and enables operation of the device. The device can also include low-power modes that allow it to operate without significantly depleting the power source while in storage and activate when used.

The device can include buttons or other controls that are actuated to turn on/off the device, put the device in/out of standby modes, initiate measurements, select modes or functions to be performed, select types of analyses, change the types of displays presented and/or their intensity or volume, clear alarms, and/or otherwise change the function of the device. These controls can include any type of control commonly used for electronic devices, such as membrane switches, optical sensors, accelerometers or movement sensors, capacitive switches, touch pads, potentiometers, optical encoding dials, pressure sensors, etc.

The device 10 can also include data storage contained in or electrically coupled to the signal processing units 62. The data storage can be carried by the circuit layer 61. The data storage can be used for recording subject signal data, analysis information and results, user actions, and/or displayed information, along with timing information, during operation. The data storage can include any type and can be stored in any type of format. For example, the data storage can be, or can include, any type of non-volatile memory system commonly used in modern electronic devices including powered RAM, one-time-programmable ROM, EPROM, EEPROM, or even consumer data storage devices such as compact flash cards, SD cards, memory sticks, etc. The data storage can include a means of encryption and/or secured access so that it is only accessible by authorized users (eg, for HIPPA compliance), including methods such as AES, Kerberos, or any other commonly used encryption and authentication standards widely used in computer and electronic devices. The data storage may also include error detection and/or correction schemes for protecting data integrity. The stored data may be accessed by wired connector or wireless links similar to those described in the programming methods.

The device can also transmit data to and/or be controlled by external systems, such as those used in monitoring systems in emergency vehicles, central monitoring stations in hospitals, mobile emergency response centers, or other situation where it may be helpful or necessary to remotely monitor the parameters or condition of one or more patients and/or the status of the monitoring device. Thus, the device can include an RF or IR transmitter 19, FIGS. 1, 7, and 18. Any of a variety of wired or wireless low and high level data exchange protocols commonly used for modern electronic communication can be used for this purpose such as LVDS, RS232, USB, Ethernet, IrDA, Bluetooth, Zigbee, 802.11, firewire, etc. The protocol can also include authentication and data encryption to secure these communications, such as AES, Kerberos, or any authentication and data security scheme commonly used in modern electronic systems for this purpose. Remotely activated controls may include any parameter that can be accessed by the user as well as additional system parameters and settings that can be only accessed by the remote system. The remote system may also include the ability to override user settings and/or transmit specific information to the device for remote display to users of specific devices. The remote system may also be capable of accessing recorded data in the system.

The device can also be capable of communicating its status, programming, settings, battery conditions, identifying information, etc, such as described above. The device can include unique identifying serial numbers and other identifying device characteristics that can be communicated as part of the programming process and/or used for inventory, determination of component or program compatibility, etc. Packages and kits that include the device can also include separate identifying information, such as ID numbers and codes, bar codes, RFID information, etc, that can be used to determine and/or verify that the device and/or its settings and programming are appropriate for the kit components.

As stated above, the device 10 can be configured as a single-use device that is disposable after use. The device can have various different configurations that limit the device to a single use. As described above, the power source 40 can be sealed within the pad or device 10 so that as the power source is depleted, the device ceases to work. Thus, the power source 40 can include a battery adapted to provide only enough power to complete a desired task. Also as described above, the tab 43 can be coupled to the release liner 52 and can extend between the power source and an electrical connection. Thus, once the pad has been prepared for use by removing the release liner, the power source is also engaged. These are examples of means for limiting the device to a single use. It will be appreciated that other means for limiting the device to a single use can be used, including for example, single-use adhesive for attaching the pad to the patient, or a circuit element that disables the device following use.

The device can also include one or more means of movement and location tracking, such as accelerometers and GPS, that are recorded and registered with the patient and device data records. These data may be used for review for general information purposes, diagnostic analysis, post-mortem analysis of the system and its functional history, and/or auditing of the history of subject condition and external events during the use of the device.

The device can be used to monitor and analyze various different physiological parameters and in various different situations. Analysis can include determination of neurological parameters and conditions, including health status, distress, neural conduction velocity, muscle tone, depth of anesthesia, alertness, level of consciousness, degree of neural injury, seizures, status epilepticus, and/or non-convulsive epileptiform activity, as well as activity indicative of imminent seizures or other neurological episodes. Analysis can also include identification of non-neurological parameters or conditions such as heart rate, breathing rate, tachycardia, bradycardia, blood oxygenation, hypoxia, etc.

The device can be used to monitor subject conditions, assist in the determination treatments to be applied to a patients in a clinical environment, and/or used in non-clinical monitoring conditions such as personal health monitoring, alertness monitoring, fitness and athletic performance monitoring, dietary guidance, training and improvement monitoring, dangerous work environments, etc.

For example, the device can be configured as a pulse oximeter, or to include a pulse oximeter. Thus, the one or more physiological sensors can include a photodiode emitter and sensor for pulse oximetry. As another example, the device can be configured to sense or monitor neural seizure or status epilepticus. Thus, the one or more physiological sensors can include a biopotential electrode.

The pads described herein are examples of means for mounting the device on the subject, and/or for carrying the various components. Other means for mounting include, for example, adhesive, mechanical clip(s), mechanical compression bands, such as armbands headbands, hair nets, etc. Thus, the entire device is completely worn on the body.

### EXAMPLE 1

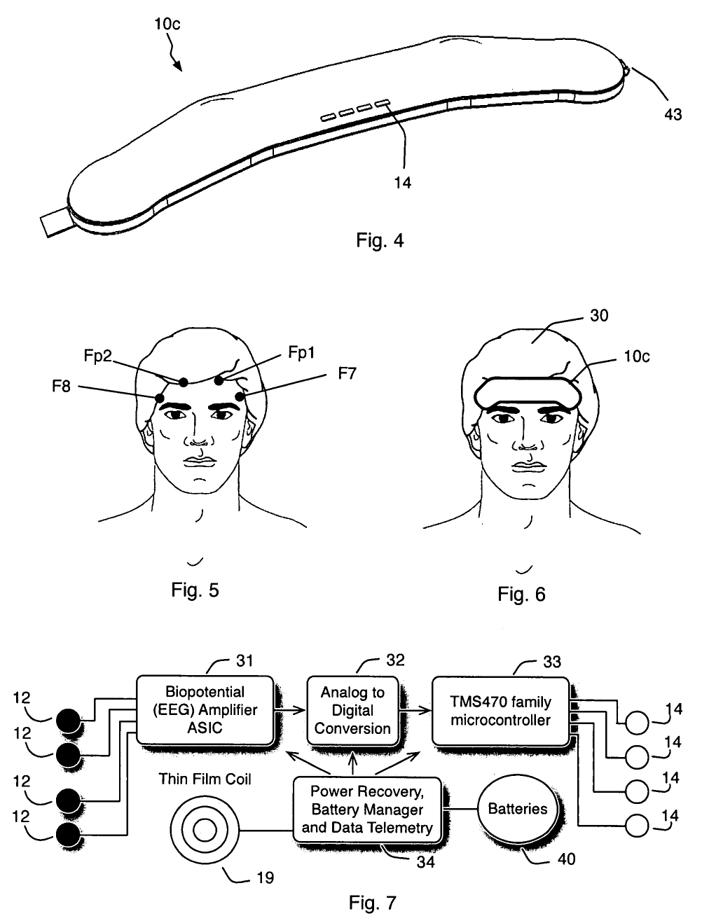

Referring to FIGS. 4-9, an exemplary embodiment of a self-contained seizure monitor device 10c to monitor a subject for an electrographic seizure is shown. Such a device can be used as a field-deployable device that can be used to monitor status epilepticus in casualties that may have been exposed to nerve agents. The device is configured as a forehead patch for detecting seizures, status epilepticus (SE), and/or other convulsive and non-convulsive epileptiform activity in subjects that may have been subjected to trauma or nerve or chemical agents. The patch configuration can be very small relative to other commercially available EEG systems, and rugged enough for robust use in field environments. The device can be similar to that described above, and the above description is herein incorporated by reference. The sensors can be, or can include, at least a pair of electroencephalographic electrodes, such as four electrodes 12, carried by the pad and spaced apart from one another, and configured to sense brain activity and generate a signal. The device can include a battery 40, surface electrodes 12, EEG acquisition and processing electronics 31, 32, and 33, and LED indicators 14 The device can be activated by removing the adhesive backing tab, and once applied, it can display seizure status for several hours as the patient is stabilized and moved to a treatment facility.

In one aspect, the device can be a small adhesive patch with integrated EEG recording and signal analysis electronics 33 that can be applied to the forehead. The patch can be activated by removing the adhesive backing (and battery contact insulator tab) and can display “OK” or “Seizure” status by small embedded LEDs and/or audible alerts. EEG biopotential amplifier chip 31 (R. R. Harrison and C. Charles, “A low-power, low-noise CMOS amplifier for neural recording applications,” IEEE J. Solid-State Circuits 38:958-965, June 2003) and low-power microcontroller technologies have progressed to the point that this type of patch design is both technically feasible and economical. In addition, with modern lithium batteries, the devices can easily have shelf lives in the range of 10 to 15 years.

The signal processing units can include a biopotential amplifier 31 to acquire EEG signals. This amplifier can have a CMOS-compatible bipolar-MOS “pseudo-resistor” to achieve low-frequency response while using capacitively-coupled inputs to reject large DC offsets. Amplifier bias currents can be selected and transistors may be sized appropriately so that the input differential pair transistors operate in the subthreshold region (i.e. weak inversion) while the other transistors operate in the traditional above-threshold region (i.e. strong inversion). By operating the input devices in subthreshold, the transconductance-to-current (gm/ID) ratio is maximized. This results in an amplifier with a nearoptimum power-noise trade-off. This amplifier has been used successfully for in vitro and in vivo electrode recordings, and a low-power multiplexers (less than 50 μW per channel) have also been added to the design and experimentally validate (5×5 mm, 32-channel IC shown at right). A complete discussion on the noise efficiency of the amplifier and EEG optimization can be found in R. R. Harrison and C. Charles, “A low-power, low-noise CMOS amplifier for neural recording applications,” IEEE J. Solid-State Circuits 38:958-965, June 2003. This fully-integrated circuit requires no off-chip components, and provides the size, power, μV noise, and bandwidth performance needed for the proposed EEG recording system.

The device can include all the necessary electronics to operate the device. The device can use a custom ASIC EEG amplifier device and a TMS470 family microcontroller 33 for program storage and data analysis. The 470 family has adequate computational power for this application and can be changed to a higher power microcontroller if necessary. The device can be battery powered during operation for a minimum of four hours. At the end of the program, an inductive link can be used, similar to an RFID reader system capable of power-up and data transfer for functional verification testing during manufacture and periodic field inspection. This inductive link can also be used to add updated software detection algorithms and updated care instructions to utilize new, improved drugs for seizure treatment for the integrated kits. The inductive link may also be used to transmit patient data to an external receiver device (a phone, a computer, a PDA, a digital audio player, or another type of external receiver) to allow a single caregiver to assess the status of a large number of patients simultaneously. The device can also have the capability to log data indicating archived patient seizure status for the duration of use. The logged data can be retrieved even after the internal battery is discharged by using an inductive power signal to activate the patch for data transfer. The electronics in the device can also include a 3-axis accelerometer to be used for adaptive motion artifact cancellation.

A simple user interface can be to provide “OK” vs. “Seizure” LED indicators. In addition, if the devices are to be stored for some time, the devices can have an initial indicator that the device is electrically functional. Furthermore, the device can be capable of communicating that the electrodes are in good contact during use. The “good connection” indicator would also be helpful as it may take a few seconds for the device to provide a reliable indication, and in an emergency situation, the LEDs might never go off as this may be interpreted as a device failure.

For example, the device can include four indicator LEDs 14, including: Power, Connected/Analyzing, green “OK”, and red “Seizure”. Although the interface could use fewer LEDs (eg, use different colors for the same LEDs to denote different states), the use of simple, single-state indicators can be unambiguous, more reliable, and non-confusing for color-blind individuals ( 1/20th of the general male population). Only one LED can be active at any one time. Other alternatives are possible for user interfaces for this device depending on how the device is packaged with drugs and other emergency response components and the degree to which classification of different ictal patterns is useful.

The device can have a seizure status indicator mounted on the outside of the device. This indicator can reflect the result the analysis of the seizure detection algorithm to the first responder. It can include a series of LEDs illuminated above descriptive text. A possible manifestation of this system may be a series of four LEDs, one to indicate the patch is powered, another to indicate sufficient electrode contact and data analysis, another LED can indicate non-ictal activity, and the fourth can signal a seizure. Only one light at a time can be turned on to simplify interaction with the device. The patch may be configured such that one light is always illuminated to avoid possible confusion. This system of LEDs can also incorporate other LED to signal first responders to administer certain drugs (e.g. two LEDs would indicate the use of either Drug A or Drug B). There may be an additional system to indicate the severity of the detected seizure. The device may also have a miniature LCD screen on the front to display a channel(s) of raw EEG data to allow trained users to more closely monitor a patient. The indicator can also have a sound signal.

For example, if the device is only used in first responder kits with auto-injectors with different drug options depending on early stage seizures vs. later stage SE EEG activity, it can be beneficial for the device to have action-based indicators, such as: Power, Patient OK, Inject Drug A, Inject Drug B, and Apply Patch More Tightly. Alternatively, if feedback from the device will be used with a more skilled technician who will also be weighing in physical symptoms to determine treatment, the device can have graded indicators of seizure activity, such as: Fasten Electrodes, and Seizure Index: Low, Med, Hi.

In another embodiment, a full graphical display may be used to indicate the current pathology status as well as its evolution over time to assist in the assessment of the effectiveness of an administered treatment, for example.

The device can include all necessary electrodes and electronics to detect EEG signals, analyze EEG signals, and display seizure status. The device can have different configurations depending on the expected skin access of the subject. For example, the device can have a two-electrode configuration, described above, or 4 lead system with three differential views across the forehead F8-Fp2, Fp2-Fp1, and Fp1-F7 (according to the international 10-20 electrode montage system) plus a central forehead reference/ground electrode (e.g. Fz), or a six lead (plus ground/reference) system which also adds electrodes that wrap around to A1 and A2 skin areas located on or behind the ear. The device can be configured to place the electrodes 30c-f on the scalp below the hairline. Electrodes may be placed at the standard EEG recording locations including, but not limited to Fp1, Fp2, F7, and F8, as shown in FIG. 8. The device can also include electrode tabs applied to the back of the neck or tabs electrodes designed to penetrate through the hair to make contact with one or more scalp sites such as the apex of the head. Electrodes can penetrate the hair by use of an electrolytic gel or sharp contacts that penetrate and hold the skin of the scalp.

The device can record brain signals from the series of electroencephalographic (EEG) electrodes 12 attached to the scalp outside the hairline. These EEG signals can be interpreted via a small, integrated circuit embedded within the patch. The circuit can analyze the data using specialized detection algorithms and display the patient's seizure status on the front of the patch.

The device can include of a series of layers including a top polymeric, such as acrylic, cover with a seizure status indicator and device labeling. The bottom of this acrylic layer can have an adhesive backing to attach it to the subsequent circuit layer. The circuit layer can be made of a flexible, thin-film polyimide (Kapton) circuit substrate. This circuit layer can include all the necessary electronics in the patch. The circuit layer can be disposed on top of a double-sided pressure sensitive adhesive foam to hold the patch close to the skin. During storage this three layered patch can have an adhesive cover over the foam layer to protect the electrodes and isolate the battery to prevent the device from powering up.

The device can use seizure detection algorithms to interpret patient EEG data. Unlike other commercially available EEG recorders, this device can selectively detect certain types of seizures. In one aspect, the device can be used to detect ongoing secondary generalized nonconvulsive seizures resulting from nerve agent exposure. Initial seizures following nerve agent exposures can be easy for non-physician first responders to diagnosis and treat. The subsequent recurring seizure activity can be more subtle, although it may still result in potentially dangerous neural sequelae. This recurring seizure activity has been identified as having similar electroencephalographic characteristics to status epilepticus (SE). Thus, the seizure detection algorithm can specifically detect SE in nerve agent victims using a combination of threshold detection and spectral decomposition elements to robustly detect seizure.

### EXAMPLE 2

Referring to FIGS. 2-3, another embodiment of a self-contained electrographic activity monitor 10b is shown which is similar in many respects to that described in Example 1 and the above description is incorporated by reference. The device integrates electrodes 12 to collect electrophysiological signals and LED sensors (not shown) for pulse oximetry and heart rate monitoring (sensors not shown). Such a device can be used as a field-deployable device to monitor the development of status epilepticus in casualties that may have been exposed to nerve agents, for example. Other applications are possible, such as neonatal epilepsy and SIDS (sudden infant death syndrome) monitoring, for example. The analysis results are displayed as a time series on a graphical display 18 to convey the effectiveness of treatment, for example. The results of pulse oximetry and heart rate monitoring are displayed on a numerical display 16b. A speaker 17 is included to indicate escalations of risk factors. The device is applied adhesively. The patch 13b is capable of flexing and conforming to the anatomy.
Seizure Detection Algorithms

Detection of seizure or ictal states from surface EEG recordings is a complex subject with a large body of literature spanning the last few decades (S. Faul, G. Boylan, S. Connolly, L. Marnane, G. Lightbody, “An evaluation of automated neonatal seizure detection methods,” Clin. Neurophysiol. 116(7):1533-41, 2005). Any existing EEG seizure detection algorithm that can be integrated into a compact, low-power microprocessor can be used with this device. Most of the first generation circuits for seizure detection were simple devices that looked for energy in certain frequency bands beyond programmed thresholds (T. L. Babb, E. Mariani, P. H. Crandall, “An electronic circuit for detection of EEG seizures records with implanted electrodes,” Electroencephalogr. Clin. Neurophysiol. 37(3):305-8, 1974). These systems were effective at detecting large seizures, but they had poor rejection of motion artifacts and other noise sources that would cause false positives. Modern algorithms developed over the last two decades generally use a combination of spectral decomposition of the EEG signal, combined with statistical metrics trained from seizure and non-seizure recordings. Some also use abstract statistical measures of the signal coherence and/or complexity.

The system can use the algorithm developed by Gotman (J. Gotman, “Epileptic recognition of epileptic seizures in the EEG,” Electronencephalogr. Clin. Neurophysiol. 54(5):530-40, 1982), and the more recent algorithm by Saab and Gotman (M. E. Saab, J. Gotman, “A system to detect the onset of epileptic seizures in scalp EEG,” Clin. Neurophysiol. 116(2):427-42, 2005), as well as variations of the “Reveal” algorithm developed by Wilson et al (S. B. Wilson, M. L. Scheuer, R. G. Emerson, A. J. Gabor, “Seizure detection: evaluation of the Reveal algorithm,” Clin. Neurophysiol. 115(10):2280-91, October 2004). The original algorithm by Gotman is commonly regarded as a gold standard for evaluating other algorithms and it is available in most EEG analysis packages. It basically looks at the strength of prototypical features of ictal activity compared to measures of the background activity. The Reveal algorithm is a more modern spectral algorithm expected to be more accurate for periodic discharges typical of ongoing status epilepticus.

A field EEG system used to assess the chemical exposure threat of nerve agent patients should be able to classify three qualitatively distinct patterns of EEG activity including primary generalized “grand mal” seizure activity accompanied by either tonic-clonic behavior or flaccid paralysis, ongoing primarily and secondarily generalized convulsive and nonconvulsive status epilepticus, and normal post-ictal patterns which may be accompanied by unrelated spastic muscle twitch.

In the case of primary generalized grand mal seizure type activity a patient will likely present a number of other pathological signs that can be interpreted by a non-clinician first responder (e.g. tonic-clonic behavior) to prompt initial drug treatment. However, patients may also exhibit flaccid paralysis during this type of seizure event making it more difficult for the non-physician to interpret. Designing an algorithm to detect seizure activity from these signals will rely on spectral shift analysis (predominance of 3 Hz activity), signal amplitude increase, and an increase in synchronous activity across recording channels. This type of seizure activity will be relatively easy to detect from EEG recordings.

Status epilepticus (SE) EEG patterns are not as easily discerned as primary generalized seizure activity. SE may present as partial or generalized epileptiform activity. Treiman (D. M. Treiman, “Generalized convulsive status epilepticus in the adult,” Epilepsia, 34 Suppl 1:S2-11, 1993) describes a succession of electrographic events which characterize SE starting with discrete seizures with low voltage fast activity. As the seizure develops, the low voltage activity spreads and gradually increases in amplitude and decreases in frequency. Cerebral rhythms are then obscured by the characteristic muscle artifact of tonic convulsive activity, which is rhythmically interrupted as the patient converts to clonic seizure activity. At this point, there is a gradual increase in amplitude and decrease in frequency until the clonic activity and its associated EEG discharged abruptly stop. Low voltage slow activity is then seen. In nerve agent induced seizure recorded in animals, this abrupt stop in high amplitude activity is seen in experiments in which animals are treated with atropine. If untreated this activity may persist for extended periods of time. There may be a gradual evolution toward consciousness during this interictal stage. However if the patient and EEG do not fully recover before the next seizure occurs, the patient is considered to be in generalized status epilepticus.

If secondary status epilepticus is allowed to persist untreated or inadequately treated, the discrete electrographic seizures begin to merge together so that there is a waxing and waning of ictal discharges on the EEG. Waxing and waning of ictal rhythms is characterized principally by a speeding up and slowing down of the frequencies of the EEG, but there may be some amplitude variability as well. As the discrete seizures merge together, the record becomes fairly continuous. The continuous discharges are then punctuated by periods of relative flattening that lengthen as the ictal discharges shorten until, finally, the patient is left with periodic epileptiform discharges on a relatively flat background. This periodic ictal firing can present as either a polyspike wave form or a simpler periodic epileptiform discharge (PED). This polyspike activity is an example of generalized convulsive SE in which patients may be either conscious or comatose. This specific example of repetitive polyspike activity was recorded from a comatose myoclonic SE patient. PED signals are spikes that occur every 1 to 2 seconds. The complexes often consist of sharp waves that may be followed by a slow wave. The question of whether or no PEDs represent interictal or postictal activity remains a topic of contemporary investigation. It has been claimed (A. Krumholz, “Epidemiology and evidence for morbidity of nonconvulsive status epilepticus,” J. Clin. Neurophysiol, 16:314-23, 1999, E. Niedermeyer and M. Ribeiro, “Considerations of nonconvulsive status epilepticus,” Clin Electroencephalogr. 31:192-5, 2000) that these complexes do not reflect ongoing seizure activity, instead they are a manifestation of damage from severe brain injury. It has also been claimed that PEDs represent ictal EEG discharges as these complexes can be eliminated with antiepileptic drugs (D. M. Treiman, “Generalized convulsive status epilepticus in the adult,” Epilepsia, 34 Suppl 1:S2-11, 1993).
Nerve Agent Exposure and Device Use Profile

Newmark (J. Newmark, “Nerve Agents,” Neurol Clin, 23:623-641, 2005) has provided several reviews of nerve agent symptoms and casualty management. Several aspects of nerve agent management have been identified that are important to this application and not obvious from a uniquely EEG monitoring perspective.

Nerve agent intoxication emergencies may unfold over the course of several minutes to as long as an hour. Depending on the methods of exposure, nerve agent symptoms may emerge quickly (e.g., inhalation or large skin contact areas) or surprisingly slowly. Of particular concern are clothing and/or skin exposures where contaminated clothes or fatty skin may act as reservoirs that continually dose the patient for some time after exposure.

EEG may actually not be very useful for patients presenting with flaccid paralysis. Patients that have systemically paralyzing levels of exposure are usually severely affected by the exposure to a degree that nerve agent symptoms are obvious, and circulatory and breathing management will be the primary goals for first responders. Patients presenting with these systems will quickly be given anticonvulsive and antiagent drugs as part of their initial treatment and EEG screening would not significantly improve patient outcomes or alter care in these extreme cases.

Early treatment and seizure management significantly improves patient outcomes. In exposure patients where the initial encounter is non-lethal, it is important to monitor for the emergence of continual seizure or status epilepticus (SE) brain activity and aggressively treat this condition quickly to avoid CNS damage and sequelae. Secondary Generalized SE in these patients will usually progress to recruit the entire cortex and result in patient death if left untreated.

Most patients with nerve agent intoxication and SE will not be completely paralyzed. This will be the case in patients with moderate levels of exposure and these patients will have outwardly visible convulsive activity that will trigger the use of anticonvulsive and anti-agent drugs in their treatment without the need for EEG monitoring.

The device can be used to manage patients between initial treatment and arrival at a treatment facility with more sophisticated monitoring. Depending on exposure type, patients may relapse into nonconvulsive or “subtle” SE and/or their fatigue may prevent convulsive activity from being readily noticed by care staff. However, recognition of SE in patients during this phase can be critical for additional anticonvulsive treatments to be administered and patients to have favorable outcomes. Once a patient is at a treatment facility, they can be analyzed with multi-lead EEG systems rather than forehead-only designs to provide more complete monitoring.

The device can be optimized for SE and nerve agent related seizures, as opposed to general clinical seizures. There are a large number of algorithms reported for general seizure detection and new ones are published every day claiming improved efficacy. Most try to detect multiple types of clinically encountered seizures and they are normally optimized for event detection during long-term monitoring. However, the present device may not have time to collect extensive background data prior to being presented with ictal activity. As such, it can be optimized specifically for nerve agent SE and post-treatment ictal activity and it can have extensive validation with nerve agent exposure model data.

Treatment protocols for these patients and appropriate SE detection algorithms are an area of active research and they will continue to evolve over the next few decades. Because of this, the device can be field upgradeable to continually improve the standard of care and protect device investments for emergency response agencies. The device can also be used in or in conjunction with treatment and casualty response kits. For example, for the particular drug injectors and algorithms used in these treatment packs, the device can be biased toward false positives or false negatives, or the labeling and indicators on the device can refer to specific user actions for the kit rather than labels for patient diagnosis.

Civilian nerve agent emergency scenes can differ from military scenes. In most civilian casualty scenes, the entire head will be accessible. As such, the device can utilize skin areas around the ears to get recordings of the temporal areas for improved cortical coverage. In addition, as a general heuristic, increasing the number of recording sites can improve the performance and robustness of seizure detection algorithms. In most civilian casualty scenes, the first responders will generally be other civilians with limited training who are using emergency response kits. As such, the kit and the EEG device can be highly algorithmic with labeling and indicators. Tradeoffs between higher sensitivity and false positives can be optimized for the specific drugs in the kit and their side effects and the expected time to be transported to a medical facility with more comprehensive EEG monitoring.
Amplifier ASIC

For the electrophysiological signal acquisition system to be very tightly integrated, ASIC biopotential amplifiers can be used. One such amplifier has been developed by Prof. Reid Harrison in the University of Utah, Department of Electrical Engineering (R. R. Harrison and C. Charles, “A low-power, low-noise CMOS amplifier for neural recording applications,” IEEE J. Solid-State Circuits 38:958-965, June 2003) This basic design has been extensively tested in animal neurophysiology experiments over the last six years, and commercial versions of the design are now being developed by Intan Technologies, LLC of Salt Lake City, Utah.

A CMOS-compatible bipolar-MOS “pseudoresistor” (Ma—Md) is used to achieve low-frequency response while using capacitively-coupled inputs to reject large DC offsets. Amplifier bias currents Ibias are selected and transistors M1-M10 are sized appropriately so that the input differential pair transistors operate in the subthreshold region (i.e. weak inversion) while the other transistors operate in the traditional above-threshold region (i.e. strong inversion). By operating the input devices in subthreshold, the transconductance-to-current (gm/ID) ratio is maximized. This results in an amplifier with a near-optimum power-noise trade-off.

This amplifier has been used successfully for in vitro and in vivo electrode recordings, and a low-power multiplexers (less than 50 μW per channel) have also been added to the design and experimentally validate (5×5 mm, 32-channel IC shown at right). A complete discussion on the noise efficiency of the amplifier and EEG optimization can be found in (R. R. Harrison and C. Charles, “A low-power, low-noise CMOS amplifier for neural recording applications,” IEEE J. Solid-State Circuits 38:958-965, June 2003). This fully-integrated circuit requires no off-chip components, and provides the size, power, μV noise, and bandwidth performance needed for the proposed EEG recording system.
Configuration Variations

Referring to FIGS. 10 and 11, a simplified device 10d is shown that is similar in many respects to those described above and the above description is incorporated herein by reference.

Referring to FIGS. 12-16, several other embodiments of a self-contained physiologic monitor are shown schematically. In FIG. 12, the monitor device 10e includes a sensor 12d enclosed in separate patch 92. The main unit 91 of the device is applied (by adhesion, for example) to the patient for convenient viewing by medical personnel and the sensor unit 92 is applied to an area that is optimal for physiological signal acquisition. In FIG. 13, the monitor device 10f is similar to 10e, the sensor unit 101 carrying the physiologic sensor 12e constitutes a clip. Alternatively, sensors may be integrated in an elastic head cap or a compressive or elastic band.

Referring to FIG. 14, the monitor device 10g is shown including multiple separate sensor units 92a as well as a separate sensor unit 92b containing multiple physiologic sensors 12f.

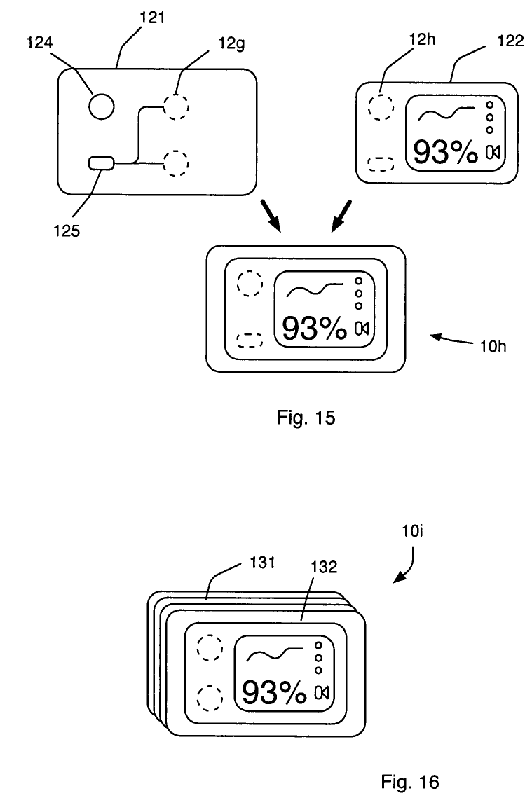

In FIG. 15, a partially reusable self-contained monitor device 10h is shown comprising a reusable portion 122 and an adhesive disposable portion 121. The disposable portion may contain disposable sensors 12g and openings 125 for sensors 12h disposed on the reusable unit 122.

In FIG. 16, a monitor device 10i with multiple adhesive layers 131 is shown to allow multiple applications of the monitor device.
Kits and Service

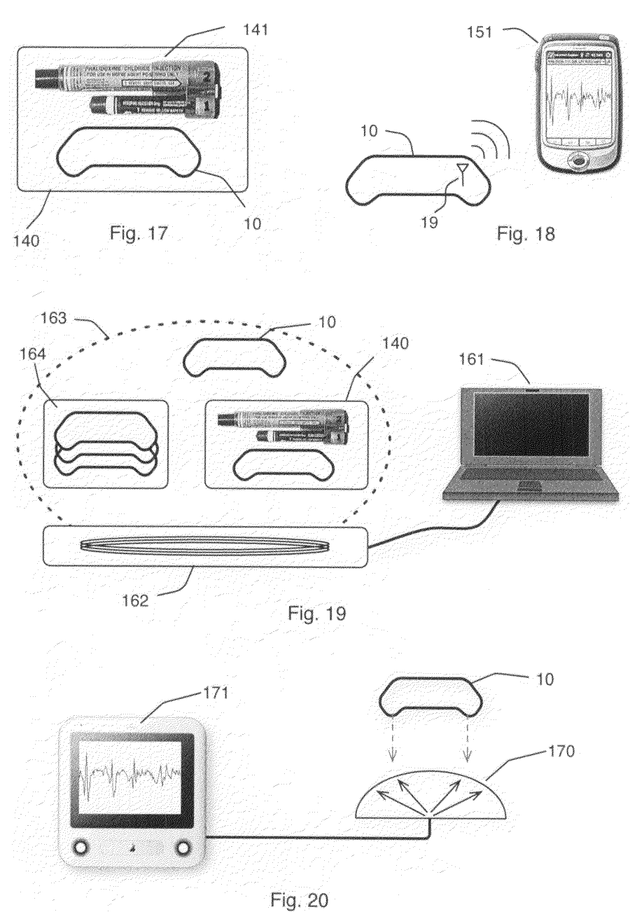

Referring to FIG. 17, the monitoring device 10 can be integrated into a complete kit 140 for non-physician first responders to use during initial treatment and transport of head trauma, brain attack, nerve agent exposure patients, or patients with other conditions to a treatment facility. The device 10 can be battery-powered and the field-deployable kit 140 can include: self-contained monitoring devices 10, treatment medication(s) 141, instruction guides, and other components. For example, the kit can include anticonvulsant and anti-cholinergic medications loaded into autoinjectors, instructions for patch use, patch indicator interpretation, and drug delivery instructions. This kit can allow an untrained person to monitor a nerve agent exposure patient for recurring ictal activity, and to treat any seizures that may occur. The patch can internally detect the presence and severity of seizure activity, and relay that information to the first responder. The patch can indicate which medication at a given dosage to administer to the patient based on recorded EEG signals. The kit can also include some electronics to inductively power the patches in order to assess remaining battery life, patch serial number, and patch operation status. This inductive link can also use low frequency power carrier modulation to send data to the device and reflect impedance telemetry to signal data back out to the programming pad 162 (FIG. 12).

Referring to FIG. 18, the monitor device 10 may establish a wireless communication with an external device such as hand-held computer 151 to upload analysis results, for example. This mechanism may be used to ensure continuity of monitoring upon transferring patients to a hospital, for example.

Referring to FIG. 19, in order to keep devices in the field properly inspected and maintained, a programming pad 162 can be used to inductively power the patch devices and query their functional status, including current battery levels. The programming pad 162 can be a standard Class-E transmitter design with low-frequency power carrier modulation to send data to the patch device and reflected impedance telemetry to signal data back out to the programming pad (similar to the method used by RFID devices used for consumer products and library books). This inductive coupling mode can allow devices to be inspected individually or within packaged kits. The inductive powering can also be used to trickle-charge the batteries for further extending shelf life.

The device may be powered by a number of different sources. An inductive coil may be placed in the storage kit to maintain charge while the patch is in storage. The device will remain charged so long as it remains in the kit, and maintain its charge for a limited duration (e.g. 4 hours) after being removed from the kit and put to use. The device can have a medical-grade single-use battery, which may be replaceable. The device may be able to transmit battery configuration information such as number of charge cycles, charge level, expected lifetime, etc. Batteries may include overcharge control means.

Referring to FIG. 20, in order to characterize and test the signal analysis systems, an additional system can be used to present simulated signals to the signal analysis device. For example, for EEG systems, scaled EEG recordings are presented onto a rubber head model 170 for device verification testing. The system can be validated by a patient simulator device 171 which transmits physiologically relevant sample EEG data to an attached patch. This patient simulator device would be made out of rubber or some other moldable nonconductive material to match the same shape as a human head. This mold would contain signal transmitters to emulate EEG signals as they might be recorded from human subjects. The emulator can include a PC connected to an analog output card and a resistor scaling network. A saved data file of archived seizure EEGs can be transmitted via this system to test the ability of the patch to detect seizure and to rapidly evaluate seizure detection algorithms without needing to use human subjects. The transmitted data can be scaled down and mixed with artifactual movement related noise to match physiological conditions.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A self-contained monitor device configured to monitor at least one physiological parameter of a subject, the device comprising:
a) a pad with an adhesive layer configured to adhere to a subject's skin;
b) at least one physiological sensor carried by the pad and configured to sense at least one subject physiological parameter and generate a physiological signal;
c) a signal processing means carried by the pad and coupled to the at least one physiological sensor for processing the physiological signal;
d) a least one indicator carried by the pad and electrically coupled to the signal processing means and configured to indicate information associated with the pad, the physiological parameter, the subject, or combinations thereof; and
e) a power source carried by the pad and electrically coupled to at least one of a physiological sensor, a signal processing means, and the at least one indicator.

2. A device in accordance with claim 1, wherein the pad is flexible and capable of contouring to a subject's body.

3. A device in accordance with claim 1, wherein the pad includes a plurality of layers stacked together, including at least: the adhesive layer, a circuit layer including the signal processing means, and a cover layer.

4. A device in accordance with claim 3, further comprising:
at least one aperture formed in the adhesive layer, and wherein the at least one physiological sensor is at least partially disposed in the at least one aperture.

5. A device in accordance with claim 1, further comprising:
means for limiting the device to a single use.

6. A device in accordance with claim 1, further comprising:
a release liner removably disposable over the adhesive layer; and
a tab coupled to the release liner and extending between the power source and an electrical connection.

7. A device in accordance with claim 1, wherein the power source is sealed within the pad.

8. A device in accordance with claim 1, wherein the at least one physiological sensor, the signal processing means, the indicator and the power source are disposed within the pad.

9. A device in accordance with claim 1, wherein the at least one physiological sensor includes a photodiode emitter and sensor for pulse oximetry.

10. A device in accordance with claim 1, wherein the at least one physiological sensor is a biopotential electrode.

11. A device in accordance with claim 1, wherein the signal processing means generates a physiological value; and wherein the indicator includes a graphical display to display the physiological value

12. A device in accordance with claim 1, wherein the signal processing means generates a physiological condition index based on at least one physiological parameter; and wherein the indicator includes a graphical display to display the physiological condition index.

13. A device in accordance with claim 12, wherein the signal processing means produces an alarm signal in response to a change of the physiological condition index.

14. A device in accordance with claim 1, further comprising:
data storage carried by the pad and electrically coupled to the signal processing means.

15. A device in accordance with claim 1, wherein the at least one physiological sensor is adapted to monitor one of at least heart rate, oxygen level, respiration rate, body temperature, cholesterol level, blood glucose level, galvanic skin response, electrophysiology, blood pressure, or combinations thereof.

16. A self-contained monitor device configured to monitor at least one physiological parameter of a subject, the device comprising:
a) a pad;
b) at least one physiological sensor carried by the pad;
c) a signal processing means carried by the pad and coupled to the at least one physiological sensor for processing a signal from the at least one physiological sensor;
d) an indicator carried by the pad and electrically coupled to the signal processing means;
e) a power source carried by the pad and electrically coupled to at least one of the at least one physiological sensor, the signal processing means, and the indicator; and
f) an adhesive disposed on the pad to secure the pad and the at least one physiological sensor to a subject's skin.

17. A device in accordance with claim 16, wherein the pad includes a plurality of layers stacked together, including at least: the adhesive layer, a circuit layer including the signal processing means and a cover layer.

18. A device in accordance with claim 16, wherein the signal processing means generates at least one physiological value; and wherein the indicator further includes a graphical display to display the at least one physiological value.

19. A device in accordance with claim 16, wherein the signal processing means generates a physiological condition index based on at least one physiological parameter; and wherein the indicator includes a graphical display to display the pathology physiological condition index; and wherein the signal processing means produces an alarm signal in response to a change in the physiological condition index.

20. A method for monitoring a physiological parameter of a subject, comprising:
adhering an adhesive layer carried by a pad of a self-contained monitor device on skin of a subject;
causing the monitor device to power from a power source carried by the pad of the self-contained monitor device, and causing at least one physiological sensor carried by the pad and to sense at least one subject physiological parameter and generate a physiological signal, and causing a signal processor carried by the pad and coupled to the at least one physiological sensor to process the physiological signal; and
perceiving an output of an indicator carried by the pad and electrically coupled to the signal processor.
